SEQUENCE LISTING (26 sequences)

SEQ ID NO: 1 - protein, 3 aa, Unknown
  fibronectin fragment
RGD

SEQ ID NO: 2 - protein, 5 aa, Unknown
  collagen fragment
KTTKS

SEQ ID NO: 3 - protein, 13 aa, Unknown
  lumican fragment
YEALRVANEVTLN

SEQ ID NO: 4 - protein, 12 aa, Unknown
  lumican fragment
EALRVANEVTLN

SEQ ID NO: 5 - protein, 4 aa, Unknown
  fibronectin fragment
RGDS

SEQ ID NO: 6 - protein, 5 aa, Unknown
  fibronectin fragment
PHSRN

SEQ ID NO: 7 - protein, 4 aa, Unknown
  fibronectin fragment
LDVP

SEQ ID NO: 8 - protein, 8 aa, Unknown
  fibronectin fragment
WQPPRARI

SEQ ID NO: 9 - protein, 3 aa, Unknown
  fibronectin fragment
IGD

SEQ ID NO: 10 - protein, 4 aa, Unknown
  fibronectin fragment
REDV

SEQ ID NO: 11 - protein, 4 aa, Unknown
  fibronectin fragment
IDAP

SEQ ID NO: 12 - protein, 15 aa, Unknown
  collagen fragment
GTPGPQGIAGQRGVV

SEQ ID NO: 13 - protein, 6 aa, Unknown
  collagen fragment
  MISC_FEATURE (3)..(3): Pyrrolysine
GRKGER

SEQ ID NO: 14 - protein, 6 aa, Unknown
  collagen fragment
GLKGEN

SEQ ID NO: 15 - protein, 6 aa, Unknown
  collagen fragment
  MISC_FEATURE (3)..(3): Pyrrolysine
GFKGER

SEQ ID NO: 16 - protein, 7 aa, Unknown
  collagen fragment
MNYYSNS

SEQ ID NO: 17 - protein, 5 aa, Unknown
  lumican fragment
EVTLN

SEQ ID NO: 18 - protein, 10 aa, Unknown
  lumican fragment
ELDLSYNKLK

SEQ ID NO: 19 - protein, 5 aa, Unknown
  laminin fragment
YIGSR

SEQ ID NO: 20 - protein, 5 aa, Unknown
  laminin fragment
IKVAV

SEQ ID NO: 21 - protein, 12 aa, Unknown
  laminin fragment
CCRRIKVAVWLC

SEQ ID NO: 22 - protein, 20 aa, Artificial Sequence
  synthetic cell adhesion motif
HSNGLPLGGGSEEEAAAVVV

SEQ ID NO: 23 - protein, 21 aa, Artificial Sequence
  synthetic cell adhesion motif
  MISC_FEATURE (21)..(21): modified lysine
HSNGLPLGGGSEEEAAAVVVX

SEQ ID NO: 24 - protein, 21 aa, Artificial Sequence
  synthetic cell adhesion motif
HSNGLPLGGGSEEEAAAVVVK

SEQ ID NO: 25 - protein, 10 aa, Unknown
  MMP enzyme cleavable sequence
TPGPQGIAGQ

SEQ ID NO: 26 - protein, 16 aa, Artificial Sequence
  synthetic lipopeptide (linker plus lumican fragment)
  MISC_FEATURE (1)..(1): C16
GGGYEALRVANEVTLN

(19) United States
(12) Patent Application Publication
(10) Pub. No.: US 2022/0296774 A1
(43) Pub. Date: Sep. 22, 2022

(54) SUPRAMOLECULAR STRUCTURES

(71) Applicant: Cellularevolution Limited, Newcastle upon Tyne (GB)

(72) Inventors: Martina Miotto, Newcastle Upon Tyne (GB); Che John Connon, Newcastle Upon Tyne (GB)

(21) Appl. No.: 17/617,730

(22) PCT Filed: Jun. 10, 2020
(86) PCT No.: PCT/GB2020/051399

§ 371 Date: Dec. 9, 2021

(30) Foreign Application Priority Data
Jun. 13, 2019 (GB) 1908522.4

(51) Int. Cl. A61L 27/22; A61L 27/54; C07K 14/78; C07K 17/08

(52) U.S. Cl. CPC A61L 27/227; A61L 27/54; C07K 14/78; C07K 17/08; A61L 2300/25

(57) ABSTRACT

The present invention relates to a supramolecular structure comprising a plurality of fused fibrils wherein each fibril comprises a plurality of cell adhesion motif lipopeptides. The invention also relates to an aqueous medium comprising said structure, a surface for cell maintenance, cell culture and/or cell bioprocessing wherein immobilised in or on the surface is said structure, as well as uses of said structure in cell maintenance, cell culture and/or bioprocessing.

### FIELD OF INVENTION

The present invention relates to a supramolecular structure comprising a plurality of fused fibrils wherein each fibril comprises a plurality of cell adhesion motif lipopeptides. The invention also relates to an aqueous medium comprising said structure, a surface for cell maintenance, cell culture and/or cell bioprocessing wherein immobilised in or on the surface is said structure, as well as uses of said structure in cell maintenance, cell culture and/or bioprocessing.

### BACKGROUND

There are a number of established and emerging commercial markets exploiting scientific advances in growing living cells to manufacture new products, including pharmaceutical drugs, stem cells, gene therapies or even cell-based meat. The goal of these products is to create an offering suitable to humanity's future needs. Such products may be more sustainable and address issues associated with scarceness of food, global warming, or access to medical treatments.

Many of these applications rely on the use of adherent cells. However, currently there are a number of challenges hampering the development of these fields. These challenges include use of animal-derived components (such as serum), which are associated with a risk of contamination, high costs and batch-to-batch variability. Use of animal products is also not compliant with Good Manufacturing Practice and often ethically controversial. Whilst serum-free media alternatives exist, these are expensive and not available for all cell types.

Furthermore, the current methods of adhesive cell culture are associated with the limitations of traditional batch cell culture. Current batch bioprocessing techniques require large surface areas and significant amounts of growth media (contributing up to 70% of overall cost), which are both a limitation to scalability. As a result, companies are not able to meet the market demand, reducing the potential of their offering, and overall slowing the development of more sustainable, cheaper, and more environmentally friendly products.

Accordingly, a product for maintaining, culturing and bioprocessing cells (including adhesive cells) more efficiently and with less potential side effects is needed. The present invention aims to provide products which at least partially address these issues.

### SUMMARY OF INVENTION

The inventors have generated a new lipopeptide supramolecular structure with a distinctive topology. This new structure has a plurality of fused fibrils, wherein each fibril comprises a plurality of cell adhesion motif lipopeptides.

The generation of this new supramolecular structure has been made possible due to a new method of making such a structure developed by the inventors. Specifically, the inventors have shown that by dissolving lyophilised lipopeptides in serum-free cell culture medium (SFM) the lipopeptides self-assemble to form fibrils that fuse together to generate a supramolecular structure that is topologically distinct from lipopeptide structures defined in the art which are generated in water. Accordingly, whilst the structures exemplified herein have been generated using SFM, it will be appreciated that such structures may also be generated using other solvents having an ionic strength that is greater than the ionic strength of water.

The inventors believe that this new structure is formed as a result of the ionic strength of the solvent in which the lipopeptides self-assemble. The inventors hypothesise that the increased ionic strength of the solvent (compared to water) generates electrostatic attraction between the lipopeptides which changes how the lipopeptides assemble.

The inventors have demonstrated that this new supramolecular structure may be formed by self-assembly of a variety of different lipopeptides. Specifically, the inventors have demonstrated this through the use of three lipopeptides that comprise a cell adhesion motif such as RGD (SEQ ID NO: 1), KTTKS (SEQ ID NO: 2) or YEALRVANEVTLN (SEQ ID NO: 3).

Surprisingly, the inventors have found that supramolecular structures comprising a plurality of fused fibrils wherein each fibril comprises a plurality of cell adhesion motif lipopeptides (such RGD, KTTKS or YEALRVANEVTLN) increase cell growth and/or cell collagen production. The inventors believe that the supramolecular structures with fused fibrils have a different bioactivity as compared to fibrillar supramolecular structures of the art and can better mimic the function of endogenous biomolecules from which part or all of the amino acid portion of the lipopeptide is obtained from.

Additionally, the supramolecular structures generated by the inventors have a higher fibril density than those structures generated in the art using water. This may be advantageous as may result in supramolecular structures with a higher density of cell adhesion motifs.

In one aspect, provided herein is a supramolecular structure comprising a plurality of fused fibrils wherein each fibril comprises a plurality of cell adhesion motif lipopeptides.

In an embodiment, the cell adhesion motif is an extracellular matrix protein sequence or a fragment or a variant thereof.

In an embodiment, the extracellular matrix protein is selected from the group consisting of fibronectin, collagen, lumican, decorin, laminin, vitronectin, fibrinogen, elastin, biglycan, heparin, tenascin and osteopontin.

In an embodiment, the cell adhesion motif is:
a) a fibronectin fragment comprising or consisting of an amino acid sequence selected from RGD (SEQ ID NO: 1), RGDS (SEQ ID NO: 5), PHSRN (SEQ ID NO: 6), LDVP (SEQ ID NO: 7), WQPPRARI (SEQ ID NO: 8), IGD (SEQ ID NO: 9), REDV (SEQ ID NO: 10), and IDAP (SEQ ID NO: 11) or a variant thereof;
b) a collagen fragment comprising or consisting of an amino acid sequence selected from KTTKS (SEQ ID NO:2), GTPGPQGIAGQRGW (SEQ ID NO: 12), GROGER (SEQ ID NO: 13), GLKGEN (SEQ ID NO: 14), GFOGER (SEQ ID NO: 15), and MNYYSNS (SEQ ID NO: 16) or a variant thereof; or
c) a lumican fragment comprising or consisting of an amino acid sequence selected from EVTLN (SEQ ID NO: 17), ELDLSYNKLK (SEQ ID NO: 18) and YEALRVANEVTLN (SEQ ID NO: 3); or
d) a laminin fragment comprising or consisting of an amino acid sequence selected from the YIGSR (SEQ ID NO: 19), IKVAV (SEQ ID NO: 20), CCRRIKVAVWLC (SEQ ID NO: 21) and RGD.

In an embodiment, the plurality of cell adhesion motif lipopeptides comprise at least two different cell adhesion motif lipopeptides.

In an embodiment, the at least two different cell adhesion motif lipopeptides are:
a) a cell adhesion motif lipopeptide comprising or consisting of KTTKS (SEQ ID NO: 2); and
b) a cell adhesion motif lipopeptide comprising or consisting of YEALRVANEVTLN (SEQ ID NO: 3).

In an embodiment, the lipopeptide comprises a lipid portion comprising a carbon chain of 6 to 24 carbon atoms.

In one aspect, provided herein is an aqueous medium comprising the supramolecular structure as described herein.

In an embodiment, the medium is a cell culture medium.

In an embodiment, the cell culture medium is serum free.

In an embodiment, the cell culture medium is Dulbecco's Modified Eagle Medium (DMEM), Ham's F12, Leibovitz's L-15 medium, RPMI-1640, Mesencult™ Basal Medium, or DMEM-F12.

In one aspect, provided herein is a surface for cell maintenance, cell culture and/or cell bioprocessing, wherein immobilised in or on the surface is a supramolecular structure as described herein.

In an embodiment, the surface is 2D or 3D.

In an embodiment, the 2D surface is a cover slip or a surface of a cell culture vessel, optionally wherein the cell culture vessel is selected from a tube, a flask, a dish or a plate comprising a plurality of wells.

In an embodiment, the 3D surface is a scaffold, optionally wherein the scaffold is a hydrogel or a polystyrene scaffold.

In one aspect, provided herein is use of a supramolecular structure, or an aqueous medium or a surface as described herein, for maintenance, culture and/or bioprocessing of a cell.

In an embodiment, the bioprocessing is for collagen production.

In an embodiment, the supramolecular structure, medium or surface promotes cell growth.

In an embodiment, the cell is selected from the group consisting of: a human stromal progenitor cell, a human adipose derived mesenchymal stem cell and an immortalised mouse myoblast cell.

Except for where the context requires otherwise, the considerations set out in this disclosure should be considered to be applicable to the structure, aqueous medium and surface in accordance with the invention, and the uses thereof.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

Embodiments of the invention are further described hereinafter with reference to the accompanying drawings, in which:

FIG. 1 shows topographical characterisation of the single-type RGDS lipopeptide self-assembled either in water (left) or serum-free medium (SFM; right) by forward deflection scan analysis performed by atomic force microscopy (AFM) of RGDS lipopeptide. Scale bars: 1 μm. Insert size: 1 μm×1 μm. The figure highlights the difference in the self-assembled structure derived from lipopeptide solubilisation in water versus SFM (the latter comprising fused fibrils as described herein).

FIG. 2 shows the topographical characterisation of single-type and composite lipopeptide systems self-assembled in serum-free media (SFM; top) or water (bottom). Forward deflection scans performed by atomic force microscopy (AFM) of RGDS, Lumican, Matrixyl and Lumican:Matrixyl lipopeptides (greyscale=500 and 100 nm, respectively). Top images scale bars: 8 and 2 μm for the image and for the inset, respectively. Bottom images scale bars: 1 and 0.5 μm for the inset, respectively.

FIG. 3 shows results of cellular biocompatibility and bioactivity assays performed on human stromal progenitor cells cultured with lipopeptides self-assembled in serum free media (SFM) or water. The graphs show human stromal progenitor cell proliferation at day 3 and 7 when using Lumican (A), Matrixyl (C) and RGDS lipopeptides (E) either solubilised in distilled water (H₂O) or in SFM at different concentrations and the amount of collagen deposited by human stromal progenitor cells after 7 days of culture with Lumican (B), Matrixyl (D) and RGDS lipopeptides (F) previously solubilised either in water on in SFM. Mean±S.D., n=3 for all experiments; *, **, *** and **** referred to statistically significant differences compared to the control (0 SFM) and corresponded to p<0.05, 0.01, 0.001 and 0.0001, respectively.

FIG. 4 shows the effect the cell culture medium has on the proliferation of RGDS-supplemented human adipose-derived mesenchymal stem cells (hASCs). Proliferation of hASCs at day 3 and 7 in the presence of RGDS lipopeptide solubilised either in SFM or in distilled water (H₂O) at 50 μM. Mean±S.D., n=3 for all experiments; **** referred to statistically significant differences compared to control in SFM and corresponded to p<0.0001.

FIG. 5 shows the effect the cell culture medium has on the proliferation of Lumican or Matrixyl-supplemented hASCs. (A) Graph reporting hASCs proliferation at day 3 and 7 in presence of Lumican or Matrixyl lipopeptides solubilised either in serum-free medium (SFM) or in distilled water (H₂O) at 50 μM using Alamar Blue assay. Graphs reporting the total amount of collagen deposited by hASCs after 7 days in culture with Lumican (B) or Matrixyl lipopeptides (C) solubilised either in SFM or in H₂O at 50 or 25 μM. Mean±S.D., n=3 for all experiments.

FIG. 6 shows how the cell culture medium in which the lipopeptide is self-assembled affects the biocompatibility and bioactivity on C2C12 cells. (A) Graph showing C2C12 proliferation at day 3 and 7 when using Lumican lipopeptide either solubilised in distilled water (H₂O) or in serum-free medium (SFM) at different concentrations. (B) Graph showing the amount of collagen deposited by C2C12 after 7 days of culture with Lumican lipopeptide previously solubilised either in H₂O on in SFM. Mean±S.D., n=1 for all experiments; *, **, *** and **** referred to statistically significant differences corresponded to p<0.05, 0.01, 0.001 and 0.0001, respectively.

FIG. 7 shows how the cell culture medium in which the lipopeptide is self-assembled affects the biocompatibility and bioactivity on C2C12 cells. (A) Graph showing C2C12 proliferation at day 3 and 7 when using Matrixyl lipopeptide either solubilised in distilled water (H₂O) or in serum-free medium (SFM) at different concentrations. (B) Graph showing the amount of collagen deposited by C2C12 after 7 days of culture with Matrixyl lipopeptide previously solubilised either in H₂O on in SFM. Mean±S.D., n=1 for all experiments; ; *, **, *** and **** referred to statistically significant differences corresponded to p<0.05, 0.01, 0.001 and 0.0001, respectively.

FIG. 8 shows how the cell culture medium in which the lipopeptide is self-assembled affects the biocompatibility and bioactivity on C2C12 cells. (A) Graph showing C2C12 proliferation at day 3 and 7 when using RGDS lipopeptide either solubilised in distilled water (H₂O) or in serum-free medium (SFM) at different concentrations. (B) Graph showing the amount of collagen deposited by C2C12 after 7 days of culture with RGDS lipopeptide previously solubilised either in H₂O on in SFM. Mean±S.D., n=1 for all experiments; ; *, **, *** and **** referred to statistically significant differences corresponded to p<0.05, 0.01, 0.001 and 0.0001, respectively.

FIG. 9 shows the topographical characterisation of the single-type RGDS lipopeptide self-assembled in (A) Leibovitz's L-15 medium, (B) RPMI 1640 medium, (C) DMEM-F12 medium, (D) Mesencult™ Basal Medium; (E) water control. It can be seen that the lipopeptides assembled in cell culture media form a supramolecular structure comprising a plurality of fused fibrils. The scale bars correspond to 2 μm.

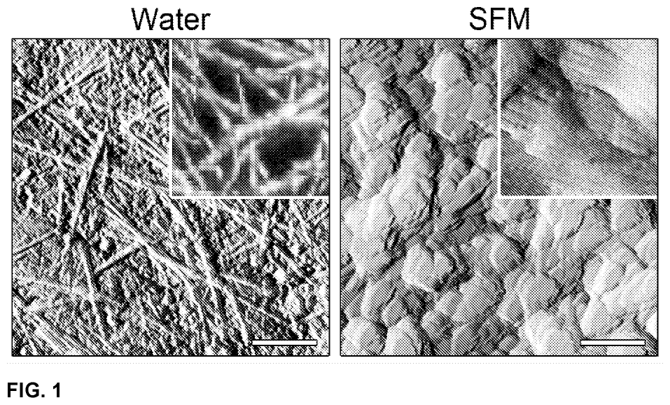

### DETAILED DESCRIPTION

Provided herein is supramolecular structure comprising a plurality of fused fibrils.

The terms “supramolecular structure” “fused fibrillar structure” or “the structure” are used herein interchangeably and refer to an aggregate composed of a plurality of fibrils. The fibrils are fused together within the supramolecular structure. Each fibril comprises a plurality of cell adhesion motif lipopeptides. The supramolecular structure may also be described as an arrangement of fibrils in which the fibrils are fused together.

It will be appreciated that fused fibrils can be identified using cryo-transmission electron microscopy (cryo-TEM), AFM, or small-angle X-ray scattering. Details of appropriate methods are provided elsewhere herein.

As used herein, the term “fibril” refers to a fibre-like structure made up of lipopeptides. The fibre-like structure (i.e. the fibril) may be a nanofiber, filament, tape, tube, twisted fibre, twisted filament, twisted tape, twisted tube, or network, or combination thereof. The structural characteristics of a fibril are well known in the art (see for example the following reviews by I. W. Hamley (Soft Matter, 2011, 7: 4122) and Stupp et al. (Faraday Discussions, 2013, 166: 9-30)).

Typically, a fibril may be in the region of about 40-290 nm wide and/or about 150-2500 nm long. The structure may be made up of uniformly and/or non-uniformly shaped fibrils. Within the structure, the fibrils may be of substantially the same size or of different size.

A plurality of fibrils present in the structure are fused together. For example, at least two, three, four, five, six, seven, eight, nine, ten, or more fibrils may be fused together to form the structure.

The fused fibrillar structure may form dense globular deposits. The globular deposits may have a diameter of at least 200 nm. For example, the globular deposits may have a diameter of at least 300, at least 400, at least 500, at least 600, at least 700, at least 800 etc nm. In one example, they have a diameter of from about 200 to about 800 nm wide.

The supramolecular structures described herein have a higher fibril density than those generated in the art using the same lipopeptides self-assembled in water. It will be appreciated that density can be determined using cryo-transmission electron microscopy (cryo-TEM) or AFM, by analysing the total area occupied by structures formed in different conditions. Details of other appropriate methods are also well known in the art.

In one example, the supramolecular structures described herein have a density of fibrils that is at least 10%, at least 20%, at least 30%, at least 40%, at least 50% higher than the density of fibrils in a supramolecular structure generated using the same lipopeptides self-assembled in water. For example, the supramolecular structures generated herein may have a density of fibrils that is at least 40% higher than the density of fibrils in a supramolecular structure generated using the same lipopeptides self-assembled in water.

As will be appreciated by a person of skill in the art, in the context of this specification, when a comparison is made to a supramolecular structure generated using water as the solvent, the water that is meant is distilled water. Accordingly, any reference to “water” solvents herein refer to distilled water.

In one example, the fibril is a nanotape. In other words, in this example, the supramolecular structure comprises a plurality of fused nanotapes.

Typically, the nanotape may be in the region of about 40-290 nm wide and/or about 150-2500 nm long. The structure may be made up of uniformly and/or non-uniformly shaped nanotapes. Within the structure, the nanotapes may be of substantially the same size or of different size.

A plurality of nanotapes present in the structure are fused together. For example, at least two, at least three, at least four, at least five, at least six, at least seven, at least eight, at least nine, at least ten, or more nanotapes may be fused together to form the structure.

The term “lipopeptide” as used herein refers to an amphiphilic molecule comprising or consisting of a lipid portion and an amino acid portion. The terms “lipopeptide”, “amphiphilic molecule”, “peptide amphiphile” and “PA” are used interchangeably herein. The amphiphilic properties enable a plurality of lipopeptides to self-assemble into the supramolecular structure. Lipopeptides are well known and their self-assembly properties are well characterised in the art (see for example Cui H. et al., Biopolymers, 2010; 94(1): 1-18). Appropriate lipopeptides may therefore easily be identified by a person of skill in the art e.g. by testing their propensity to self-assemble under certain conditions and form supramolecular structures. Lipopeptide self-assembly and corresponding c.a.c. can be evaluated by the Thioflavin (ThT) and pyrene (Pyr) fluorescence spectroscopy methods. Fluorescence spectra are recorded with a Fluorescence Spectrometer. For the ThT assay, the spectra are typically recorded from 460 to 600 nm using an excitation wavelength λₑₓ=440 nm and the lipopeptide dissolved in a 4-5 ×10⁻³% (w/v) ThT solution. For the Pyr assay, the spectra are typically recorded from 360 to 550 nm using an excitation wavelength λₑₓ=339 nm. Pyr assays are performed using a 1-1.5 ×10⁻⁵% (w/v) Pyr solution as a diluent. The Florescence intensity is plotted against a log of the lipopeptide concentration. The inflection point for the data denotes a change of environment for the ThT/Pyr molecule and is used to identify the c.a.c.

A lipopeptide nanostructure can be evaluated by cryo-transmission electron microscopy (cryo-TEM) using a field-emission cryo-electron microscope (e.g. JEOL JEM-3200FSC), AFM, or small-angle X-ray scattering. For cryo-TEM, vitrified specimens are prepared onto holey carbon copper grids with 3.5 μm hole size. A lipopeptide solution is applied to the grid and then vitrified in a 1/1 mixture of liquid ethane and propane at −180° C. The cryo-electron microscope is operated at at −187° C. during the imaging. Lipopeptide solutions are heated from −187° C. to −60° C. at ˜10-5 Pa, before being imaged at −187° C. The heating process from −187 to −60° C., equivalent to a freeze drying process in the microscope, allows for the sublimation of the ice from the sample and removes the vitrified water. Images are taken using bright-field mode and zero-loss energy filtering (omega type) with a slit width 20 eV. Micrographs are recorded using a CCD camera (e.g. Gatan Ultrascan 4000).

The term “plurality”, as used herein, is defined as two or more than two. The two or more lipopeptides in the structure may be the same or they may be different.

The amino acid portion of the lipopeptide may be a natural or synthetic amino acid sequence. A natural amino acid sequence is one that exists in nature and encodes a protein or a fragment thereof. The natural amino acid sequence may encode a human, animal, plant, fungal, Protista, Archaea, and/or bacterial protein or fragment thereof. For example, the fragment may comprise from about 3 to about 40 amino acids, such as from about 3 to about 20, or from about 3 to about 10 amino acids.

The amino acid portion may comprise an extracellular matrix protein sequence (i.e. a sequence of an extracellular matrix protein; also referred to as a motif) that is involved in cell adhesion. The amino acid portion may alternatively comprise fragments or variants of such sequences, where the fragments or variants are also involved in cell adhesion. Such sequences (including fragments and variants thereof) are referred to herein as “cell adhesion motifs”. In other words, as used herein, the term “cell adhesion motif” encompasses extracellular matrix protein motifs, and fragments and variants thereof, which are involved in cell adhesion. The term “cell adhesion motif” therefore encompasses extracellular matrix protein cell adhesion motifs, or fragments or variants thereof, where the fragments or variants are also involved in cell adhesion.

As used herein, the term “cell adhesion motif lipopeptide” therefore describes a lipopeptide that comprises an amino acid portion that comprises or consists of an extracellular matrix protein motif, or a fragment or variant thereof, that is involved in cell adhesion.

As used herein, “involved in cell adhesion” refers to promoting cell adhesion to the lipopeptide, and/or directly adhering or binding to the cell e.g. by binding to cells via cell surface molecules, such as integrins, displayed on the surface of the cells. Cell adhesion motifs are typically capable of adhering to cells directly, for example, by binding to cells via cell surface molecules, such as integrins, displayed on the surface of the cells.

Many extracellular matrix proteins involved in cell adhesion are known in the art. By way of example, an extracellular matrix protein involved in cell adhesion may be selected from the group consisting of fibronectin, collagen (such as types I, II, Ill and V), lumican, decorin, laminin, vitronectin, fibrinogen, elastin, biglycan, heparin, tenascin and osteopontin. Additionally, the cell adhesion motifs may be any peptide derived from any of the aforementioned proteins, including derivatives or fragments containing the binding domains of the above-described molecules. Example motifs include integrin-binding motifs, such as the

RGD (arginine-glycine-aspartate) motif, the YIGSR (tyrosine-isoleucine-glycine-serine-arginine) motif, and related peptides that are functional equivalents. For example, peptides containing RGD sequences (e.g., RGDS) and WQPPRARI sequences are known to direct spreading and migration properties of endothelial cells, and YIGSR peptide has been shown to promote epithelial cell attachment. Whether an amino acid sequence is a cell adhesion motif can be determined by screening peptide libraries for adhesion and selectivity to specific cell types. Cell adhesion motifs may also be developed empirically via Phage display technologies.

The amino acid portion of the lipopeptide may comprise or consist of one or more cell adhesion motifs. For example, the amino acid portion of the lipopeptide may comprise or consist of 2, 3, 4, 5 or more cell adhesion motifs (which may be in tandem or may be spatially separated e.g. by other amino acids or linkers within the amino acid portion of the lipopeptide). In an example where the amino acid portion of the lipopeptide comprises more than one cell adhesion motif, some or all of the motifs may be same. Alternatively, some or all of the motifs may be different.

The cell adhesion motif may be an extracellular matrix protein or a fragment or variant thereof that is involved in cell adhesion (i.e. an extracellular matrix protein that is involved in cell adhesion; a variant of an extracellular matrix protein, wherein the variant is involved in cell adhesion; a fragment of an extracellular matrix protein, wherein the fragment is involved in cell adhesion; or a variant of a fragment of an extracellular matrix protein, wherein the variant of the fragment is involved in cell adhesion).

The cell adhesion motif may be a fibronectin fragment that comprises or consists of an amino acid sequence selected from the group consisting of RGD, RGDS, PHSRN, LDVP, WQPPRARI, IGD, REDV, and IDAP or a variant thereof. The variant may be a conservative amino acid substitution variant e.g. having one, two or three conservative amino acid substitutions compared to an amino acid sequence selected from the group consisting of RGD, RGDS, PHSRN, LDVP, WQPPRARI, IGD, REDV, and IDAP.

Alternatively, the cell adhesion motif may be a collagen fragment that comprises or consists of an amino acid sequence selected from the group consisting of KTTKS, GTPGPQGIAGQRGVV, GROGER, GLKGEN, GFOGER, and MNYYSNS or a variant thereof. The variant may be a conservative amino acid substitution variant e.g. having one, two or three conservative amino acid substitutions compared to an amino acid sequence selected from the group consisting of KTTKS, GTPGPQGIAGQRGVV, GROGER, GLKGEN, GFOGER, and MNYYSNS.

Alternatively, the cell adhesion motif may be a lumican fragment that comprises or consists of an amino acid sequence selected from the group consisting of EVTLN, ELDLSYNKLK and YEALRVANEVTLN or a variant thereof. The variant may be a conservative amino acid substitution variant e.g. having one, two or three conservative amino acid substitutions compared to an amino acid sequence selected from the group consisting of EVTLN, ELDLSYNKLK and YEALRVANEVTLN.

Alternatively, the cell adhesion motif may be a laminin fragment that comprises or consists of an amino acid sequence selected from the group consisting of YIGSR, IKVAV, CCRRIKVAVWLC and RGD or a variant thereof. The variant may be a conservative amino acid substitution variant e.g. having one, two or three conservative amino acid substitutions compared to an amino acid sequence selected from the group consisting of YIGSR, IKVAV, CCRRIKVAVWLC and RGD.

As mentioned, amino acid portions of the lipopeptides may be synthetic. By “synthetic” it is meant that they comprise amino acid sequences that do not exist in nature. A synthetic amino acid portion may resemble an amino acid sequence, for example a peptide, that occurs in nature. Merely by way of example, a synthetic cell adhesion motif may be selected from the group consisting of V₂A₂E₂, HSNGLPLGGGSEEEAAAWV (SEQ ID NO: 22), HSNGLPLGGGSEEEAAAVVV(K) (SEQ ID NO: 23) and HSNGLPLGGGSEEEAAAVWK (SEQ ID NO: 24) or a variant thereof.

The amino acid portion may comprise an enzyme-cleavable sequence.

In one example, the amino acid portion may comprise or consist of an enzyme-cleavable sequence and a cell adhesion motif. In such an example, the enzyme-cleavable sequence may be at the N-terminal of the amino acid portion, whereas the cell adhesion motif may be at the C-terminal of the amino acid portion.

The term “enzyme-cleavable sequence” refers to an amino acid sequence that can be cleaved by an enzyme. The enzyme may be, for example, a protease. It will be appreciated that an enzyme-cleavable sequence that can be cleaved by a protease may also be referred to as a protease-cleavable sequence. Examples of proteases and sequences cleaved by proteases will be well known to those skilled in the art. Merely by way of example, a protease may be selected from the group consisting of a metalloprotease, serine protease, cysteine protease, threonine protease, aspartic protease, glutamic protease and asparagine peptide lyases. A metalloprotease may be, for example, MMP1 or MMP2. TPGPQGIAGQ (SEQ ID NO: 25) is an example of an enzyme-cleavable sequence that may be cleaved by MMP1 or MMP2.

The presence of an enzyme-cleavable sequence may be useful for enabling self-directed release of cells maintained and/or cultured on the fused fibrillar supramolecular structure disclosed herein. Cells released from the supramolecular structure by cleavage of the enzyme-cleavable sequence may be carrier-free, structurally, and/or phenotypically equivalent to their natural counterparts. Such cells may be advantageous for example in the context of autologous transplantation. By “carrier-free” it is meant that the cells are free from remnants of the supramolecular structure on which they were maintained and/or cultured.

The term “fragment” as used herein refers to a peptide that is a truncation of the corresponding wild type amino acid sequence. A fragment of the peptide may share 100% identity with the portion of the wild type amino acid sequence that it corresponds to.

As used herein, the term “variant” refers to a peptide in which one or more amino acid have been replaced by different amino acids as compared to the corresponding wild type amino acid sequence. It is well understood in the art that some amino acids may be changed to others with broadly similar properties without changing the nature of the activity of the peptide (conservative substitutions). Generally, the substitutions which are likely to produce the greatest changes in a peptide's properties are those in which (a) a hydrophilic residue (e.g., Ser or Thr) is substituted for, or by, a hydrophobic residue (e.g. Leu, lie, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

The fragment or variant of a cell adhesion protein may substantially retain the biological function of the corresponding wild type peptide. The term “biological function” as used herein may refer to the ability to promote cell binding. By “substantially retains” biological function, it is meant that the fragment or variant retains at least about 50%, 60%, 75%, 85%, 90%, 95%, 97%, 98%, 99%, or more, of the biological function of the wild type peptide to, for example, promote cell binding. Indeed, the fragment or variant may have a higher biological function than the wild type peptide. The fragment or variant may have 110%, 120%, 130%, 140%, 150%, 160%, 170%, 180%, 190%, 200%, or more, of the biological function of the wild type peptide to, for example, promote cell binding.

The lipid portion of the lipopeptide may be linear, branched or cyclic. For example, the lipid portion may be linear.

The lipid portion may comprise a hydrophobic carbon chain of 6 to 24 carbon atoms. The lipid portion may therefore comprise a carbon chain of 6, 7, 8, 9, 10, 11, 12, 13, 14, 15, 16, 17, 18, 19, 20, 21, 22, 23, 24 or more carbon atoms. For example, the lipid portion will comprise a carbon chain of 16 or 18 carbon atoms. It will be appreciated that, when a lipid portion is referred to as, for example, C16 or C18, it means that the lipid portion comprises carbon chain of 16 or 18 carbon atoms, respectively. By way of example and not limitation, the lipid portion may comprise or consist of dodecanoic acid (lauric acid), tetradecanoic acid (myristic acid), hexadecanoic acid (palmitic acid), octadecenoic acid (stearic acid), oleic acid, linoleic acid, and linolenic acid.

The lipid portion may be saturated or unsaturated.

The lipid portion and amino acid portion of the lipopeptide may be attached directly or indirectly. By attached directly, it is meant that the lipid and peptide portions are not separated by a linker. For example, the lipid and amino acid portion may be covalently coupled. By attached indirectly it meant that the lipid and peptide portions are separated by a linker.

The term “linker” as used herein refers to a moiety which is located between the lipid portion and the amino acid portion. Choosing a suitable linker is within the capabilities of those having ordinary skill in the art. For example, where a rigid linker is desired, it may be a rigid polyunsaturated alkyl or an aryl, biaryl, heteroaryl, and the like. When a flexible linker is desired, it may be a flexible peptide, such as Gly-Gly-Gly, or a flexible saturated alkanyl or heteroalkanyl. Hydrophilic linkers or spacers may be, for example, polyalcohols or polyethers, such as polyalkyleneglycols. Hydrophobic linkers or may be, for example, alkyls or aryls.

For example, the linker may be a flexible peptide, such as Gly-Gly-Gly.

In one example, the cell adhesion motif lipopeptide may comprise a C16 lipid portion and an amino acid portion comprising or consisting of the sequence RGDS. In this example, the lipopeptide may comprise a linker (e.g. a Gly-Gly-Gly linker) between the lipid and the RGDS sequence.

In another example, the cell adhesion motif lipopeptide may comprise a C16 lipid portion and an amino acid portion comprising or consisting of the sequence RGDS and an enzyme-cleavable sequence. In this example, the enzyme cleavable-sequence may be TPGPQGIAGQ. The enzyme-cleavable sequence may be at the N-terminal of the amino acid portion and the cell adhesion motif may be at the C-terminal of the amino acid portion.

In another example, the cell adhesion motif lipopeptide may comprise a C16 lipid portion and an amino acid portion comprising or consisting of the sequence YEALRVANEVTLN. In this example, a linker between the lipid portion and the amino acid portion may not be needed e.g. the lipid portion and the YEALRVANEVTLN amino acid sequence may be attached directly.

In another example, the cell adhesion motif lipopeptide may comprise a C16 lipid portion and an amino acid portion comprising or consisting of the sequence KTTKS. In this example, a linker between the lipid portion and the amino acid portion may not be needed e.g. the lipid portion and the KTTKS amino acid sequence may be attached directly.

The supramolecular structure may be single-type or composite.

A structure is composite when it is made up of non-identical molecules, wherein some or all of the molecules are lipopeptides and wherein at least one of the lipopeptides is a cell adhesion motif lipopeptide.

In one example, the composite structure comprises or consists of at least two different lipopeptides, wherein at least one of the lipopeptides is a cell adhesion motif lipopeptide. The composite structure may further comprise at least one non-cell adhesion motif lipopeptide. A non-cell adhesion motif lipopeptide is one that does not comprise an amino acid sequence that promotes cell binding. Composite structures may be comprised by different lipopeptides evenly interspersed, or separated in well-defined, discrete domains of the structure.

In one example, the at least one cell adhesion motif lipopeptide may comprise a C16 lipid portion and an amino acid portion comprising or consisting of the sequence YEALRVANEVTLN. The adhesion motif lipopeptide may also further comprise a Gly-Gly-Gly linker between the lipid portion and the amino acid portion (e.g. there may be a Gly-Gly-Gly linker between the lipid portion and the YEALRVANEVTLN sequence).

The at least one non-cell adhesion motif lipopeptide may be used to dilute cell adhesion motif lipopeptides that form the supramolecular structure, for reduced steric hindrance.

In one example, the composite structure may comprise or consist of at least two different lipopeptides, wherein the at least two lipopeptides are both (distinct) cell adhesion motif lipopeptides. For example, one of the at least two cell adhesion motif lipopeptides may comprise a C16 lipid portion and an amino acid portion comprising or consisting of the sequence YEALRVANEVTLN. Such a lipopeptide may be referred to as “C₁₆-YEALRVANEVTLN”. The adhesion motif lipopeptide may further comprise a Gly-Gly-Gly linker between the lipid portion and the amino acid portion. Such a lipopeptide may be referred to as “C₁₆-GGG-YEALRVANEVTLN”. The second of the at least two cell adhesion motif lipopeptides may comprise a C16 lipid portion and an amino acid portion comprising or consisting of the sequence KTTKS. Such a lipopeptide may be referred to as “C₁₆-KTTKS”.

It will be appreciated that in a composite structure, the ratio of the different lipopeptides may vary. For example, where the structure comprises two different types of lipopeptides, the molar ratio of the first lipopeptide to the second lipopeptide may be from about 1:10 to about 10:1, from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or about 1:1. For example, the ratio of the first lipopeptide to the second lipopeptide may be from about 1:7 to about 7:1, from about 1:6 to about 6:1, or from about 1:5 to about 5:1.

A structure is single-type when it is made up of identical lipopeptide molecules.

In an embodiment, where the structure is single-type, the structure may comprise a cell adhesion motif lipopeptide comprising a C16 lipid portion and an amino acid portion comprising a cell adhesion motif selected from the group consisting of RGDS, EALRVANEVTLN and KTTKS. In one example, when the amino acid sequence is RGDS the cell adhesion motif lipopeptide may further comprise a Gly-Gly-Gly linker between the lipid portion and the RGDS sequence.

In an embodiment, where the structure is single-type, the structure may comprise a cell adhesion motif lipopeptide comprising a C16 lipid portion and an amino acid portion comprising a cell adhesion motif selected from the group consisting of RGDS, EALRVANEVTLN and KTTKS. In one example, when the amino acid sequence is RGDS the amino acid portion may further comprise an enzyme-cleavable sequence (for example TPGPQGIAGQ). The enzyme-cleavable sequence may be at the N-terminal of the amino acid portion and the cell adhesion motif may be at the C-terminal of the amino acid portion.

The inventors have found that the fused fibrillar supramolecular structure described herein is formed from lipopeptides when they self-assemble in an aqueous medium having an ionic strength that is greater than the ionic strength of distilled water. Surprisingly, however, once the fused fibrillar supramolecular structure is formed, the structure maintains its topology even if placed in a different medium (e.g. a medium having a lower ionic strength than the solvent in which the structure self-assembled, such as water).

Accordingly, an aqueous medium is provided herein which comprises a fused fibrillar supramolecular structure as described herein. As stated previously herein, the fused fibrillar supramolecular structure comprises a plurality of cell adhesion motif lipopeptides.

The term “aqueous medium” as used herein refers to any liquid medium containing water. The aqueous medium may be cell culture medium, phosphate-buffered saline (PBS) or other saline solutions, or may, in fact, be water. However, it will be appreciated that the term “aqueous medium” does not imply that water should always be the major constituent of the medium. The aqueous medium may be serum free.

The terms “cell culture medium” and “culture medium” (plural “media” in each case) refer to a nutritive solution for cultivating live cells and may be used interchangeably. The cell culture medium may be a complete formulation, i.e., a cell culture medium that requires no supplementation to culture cells, or may be an incomplete formulation, i.e., a cell culture medium that requires supplementation or may be a medium that may supplement an incomplete formulation or in the case of a complete formulation, may improve culture or culture results.

Various cell culture media will be known to those skilled in the art, who will also appreciate that the type of cells to be cultured may dictate the type of culture medium to be used.

Merely by way of example and not limitation, the culture medium may be selected from the group consisting of Dulbecco's Modified Eagle's Medium (DMEM), Ham's F-12 (F-12), Leibovitz's L-15 medium, RPMI-1640, Mesencult™ Basal Medium, Minimal Essential Medium (MEM), Basal Medium Eagle (BME), Ham's F-10, αMinimal Essential Medium (αMEM), Glasgow's Minimal Essential Medium (G-MEM), and Iscove's Modified Dulbecco's Medium (IMDM), or any combination thereof. Other media that are commercially available (e.g., from Thermo Fisher Scientific, Waltham, Mass.) or that are otherwise known in the art can be equivalently used in the context of this disclosure. Again, only by way of example, the media may be selected from the group consisting of 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's modified MEM, IPL-41 insect cell medium, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, and KNOCKOUT D-MEM, or any combination thereof.

The cell culture medium may be serum-free. For example, the serum-free medium may be DMEM, F-12, Leibovitz's L-15 medium, RPMI-1640, Mesencult™ Basal Medium, or a combination thereof (for example DMEM-F12).

The aqueous medium (for example serum free medium such as DMEM, F-12, Leibovitz's L-15 medium, RPMI-1640, Mesencult™ Basal Medium, or DMEM-F12) may comprise the fused fibrillar supramolecular structure at a concentration from about 1 μM to about 100 μM. Suitably, the aqueous medium may comprise the fused fibrillar supramolecular structure at a concentration from about 5 μM to about 50 μM, from about 10 μM to about 30 μM, or from about 12 μM to about 25 μM. For example, the aqueous medium may comprise the fused fibrillar supramolecular structure at a concentration of about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, or more. The skilled person will be able to determine a suitable concertation, for example by analysing the medium's effect on cell proliferation or effect on cell bioprocessing (such as collagen production). Exemplary methods for analysing cell proliferation and collagen production are provided elsewhere in the present specification.

A surface for cell maintenance, cell culture and/or cell bioprocessing is also provided herein. Immobilised in or on the surface is a fused fibrillar supramolecular structure as described herein.

The term “surface” as used herein refers to an area on which cells may be grown. The surface can be 2-dimensional (2D) or 3-dimensional (3D). An example of a 2D surface is a cover slip, or a surface of a culture vessel, such as a tube, a flask, a dish or a plate comprising a plurality of wells. The culture vessel may be a glass, plastic, or metal container that can provide an aseptic environment for culturing cells. An example of a 3D surface is a scaffold, such as a polystyrene scaffold (eg. Alvetex™) or a gel scaffold (eg. hydrogel).

As mentioned, the fused fibrillar supramolecular structure may be immobilised on the surface so as to provide a surface that is coated with the supramolecular structure. The surface may be coated partially or completely. Methods of coating surfaces with supramolecular structures are generally known in the art. By way of example, a surface may be coated by drop-spotting on the surface and homogenously distributing a solution comprising the lipopeptides, followed by drying the surface to form a thin film of self-assembled fused fibrillar supramolecular structure. In an example, the structure may be immobilised on a 2D surface, such as a cover slip, or a surface of a culture vessel such as a tube, a flask, a dish or a plate comprising a plurality of wells.

The fused fibrillar supramolecular structure may be in the surface for cell maintenance, cell culture and/or cell bioprocessing. By “in the surface” is meant that the structure is incorporated into the surface, such that it is partially or fully encapsulated by the surface. Methods for incorporating into a surface a supramolecular structure are also known in the art. For example, the lipopeptides which form supramolecular structure the may be added to the solution from which the surface (such as a 3D scaffold) is made from.

It will be appreciated that in some examples, the fused fibrillar supramolecular structure described herein may, in fact, itself be the surface for cell maintenance, cell culture and/or cell bioprocessing. In such an embodiment, the structure may be in an aqueous medium or may be immobilised in or on the surface. An embodiment in which the aqueous medium comprises the structure and wherein the structure is the surface may be particularly advantageous in the context of adherent cells, where the surface area may be a limiting factor for cell growth. The fused fibrillar supramolecular structures in the solution may provide a greater surface area for cell growth than, for example, the surfaces of the cell culture vessel containing the aqueous medium. This may have advantages such as reduction of cost associated with cell culture and/or improved bioprocessing. The structure may be a 3D surface.

The term “cell maintenance” as used herein refers to keeping the cells alive in an artificial (e.g., an in vitro) environment without substantially increasing the cell number. The term “cell culture” as used herein refers to keeping the cells in an artificial environment under conditions favouring growth, differentiation, and/or continued viability of the cells. In the context of the present disclosure, the cell can be an individual or a population of cells, or a tissues, organ or organ system. The cell may be eukaryotic (e.g., animal, plant and fungal cells) or prokaryotic (e.g., bacterial cells). The cell may be an animal cell. For example, the cell is mammalian (for example human or mouse). Merely by way of example, a human cell may be a human stromal progenitor cell or a human adipose derived mesenchymal stem cell. Merely by way of example, a mouse cell may be immortalised mouse myoblast cell.

The aqueous medium, the surface, or indeed the structure described herein may promote cell growth. Cell growth may be promoted for example if cell number and/or cell viability is increased as compared to a suitable control.

The term “cell bioprocessing” as used herein refers to producing a molecule of biological origin. The aqueous medium, the surface, or indeed the fused fibrillar supramolecular structure described herein may improve bioprocessing of cells. By improve cell bioprocessing it is meant that cells maintained or cultured in the presence of the fused fibrillar supramolecular structure described herein may produce more of the molecule of biological origin as compared to a suitable control. The molecule of biological origin may be, for example, collagen.

Methods of cell culture are well known to those skilled in the art. In the context of the present specification, cell culture may be for as long as required to obtain a required effect, for example increased cell production of a molecule of biological origin, such as collagen. Merely by way of example cell culture in the presence of a fused fibrillar supramolecular structure as described herein may be for about 3 hours, about 4 hours, about 5 hours, about 6 hours, about 7 hours, about 8 hours, about 9 hours, about 10 hours, about 12 hours, or more. Suitably, cell culture may be for about 24 hours (about 1 day), about 36 hours, about 48 hours (about 2 days), about 60 hours, about 72 hours (about 3 days), or more. Suitably, cell culture may be for about 84 hours, about 96 (about 4 days) hours, about 108 hours, about 120 hours (about 5 days), about 132 hours, about 144 hours (about 6 days), about 156 hours, about 168 hours (about 7 days), about 180 hours, about 192 hours (about 8 days), about 204 hours, about 216 hours (about 9 days), or more.

Suitably, cell culture may be from about 1 day to about 9 days, for example from about 2 days to about 8 days, or from about 3 days to about 7 days.

A suitable control may be for example cells grown without the presence of the fused fibrillar supramolecular structure described herein. The control cells may be grown in the presence of a fibrillar supramolecular structure of the art such as a structure that has self-assembled in water.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Various further aspects and embodiments of the present invention will be apparent to those skilled in the art in view of the present disclosure.

### EXAMPLES

1. Materials and Methods
1.1 Preparation of Lipopeptide Solutions in Serum Free Medium

Lyophilized lipopeptides were weighed with static eliminator turned on. Lyophilized lipopeptides are highly electrostatic thus the powder transfer is compromised without static eliminator. This is an essential requirement to not loose lipopeptide s powder and precisely weight the required amount of lipopeptides.

Lipopeptides were then solubilized in serum-free medium at the desired concentration. The serum-free medium is DMEM-F12, Leibovitz's L-15 medium, RPMI 1640 medium, or Mesencult™ Basal Medium supplemented with 1 mM ascorbic acid and 1:100 insulin-transferrin-selenium. Stock solutions must be prepared to have the c.a.c. value greater than the c.a.c. value of lipopeptide used. C.a.c. value varies depending on the lipopeptide sequence and is determined via pyrene fluorescence measurements.

Exemplary stock: 1.52 mM for RGDS lipopeptide and 1.25 mM for Lumican:Matrixyl lipopeptides (the latter consists of mixing together the 2 powders at the ratio 15:85 to obtain a final concentration of 1.25 mM). Solution vortexed for 30-45 minutes at room temperature then sonicate at 55° C. for 30 min. Solution should be rotated overnight at 4° C. If the lipopeptides are still not well solubilized, repeat steps vortexing and sonication. The final stock solution needs to be substantially, preferably completely transparent and without lipopeptide aggregates. Stock should be stored at 4° C.
1.2 Preparation of Lipopeptide Solutions in Water
1) Weight the lipopeptides.
2) Dissolve the lipopeptides in aqueous solution at the desired concentration, above the critical aggregation concentration (c.a.c.)
3) Vortex the solution for at least 15 minutes
4) Sonicate the solution for at least 30 min and not above 55° C.
5) Rotate the solution overnight in a cool environment.
6) Store the final stock solution in a cool environment.
1.3 Cell Proliferation

Cell proliferation can be evaluated via Alamar Blue assays, with cell number extrapolated from standard calibration curves using known number of cells for each experiment. Cells are incubated at 37° C. with resazurin reagent at 50 μM (Sigma Aldrich), prepared in 1:10 dilution using fresh culture medium, for 5-3 h, after which 100 μl of culture supernatants (in triplicate) are sampled for fluorescence emission analysis at 590 nm using either a Fluoroskan Ascent fluorescent spectrophotometer or a Varioskan™ Lux (both from Thermo Scientific). The incubation time differ depending on the experiment and on the cell type.
1.4 Collagen Deposit Assay

The amount of collagen deposited by the cells is investigated using the Sirius Red assay (Jimenez et al., 1985). In specific, cells are fixed in 70% ice-cold ethanol and transferred to −80° C. for at least 10 min to ensure the complete fixation of the material. Subsequently, the ethanol solution is removed and each well was gently washed with distilled water. Cells are treated with Sirius-red/picric acid solution (Sigma Aldrich) and incubated overnight at 4° C. with gentle agitation. The following day, any unbound dye is removed and cells were gently rinsed with distilled water. Cells are then treated with 500 μl of 1M NaOH (Fisher Scientific) at room temperature for 10 min under agitation to disperse the solution and 100 μL aliquots of each sample were then transferred in triplicate to a 96 well plate. The total collagen is calculated by comparing the absorbance of the resulting samples at 490 nm, read using either a Multiskan Ascent™ or a Varioskan™ Lux (both from Thermo Fisher Scientific), to that of known standard concentrations of collagen. Each tested condition is performed in triplicate.
1.5 Atomic Force Microscopy (AFM)

Analysis of materials surface topography are performed using a Nanosurf Easyscan 2-controlled atomic force microscope equipped with ContAI-G soft contact mode cantilevers (BudgetSensors; Bulgaria) with a resonant frequency of 13 kHz and nominal spring constant of 0.2 N/m. Briefly, the different samples are mounted on parafilm-covered glass slides (Bemis; USA and Thermo Fisher Scientific, respectively) to minimise sample displacement and drift. Surface topography is analysed from three separate regions in each sample, with 512×two-direction lines scanned at 10 μm/s, at 1 nV, and with P- and I-gains of 1. Topographic data is processed for line wise and tilt correction using the Scanning Probe Image Processor software package (Image Metrology A/S). Data is analysed using the OrientationJ plugin from ImageJ (open source Java application NIH, USA) v1.46 for measuring dimensions and distribution of specimens. All experiments were performed on four individual areas in each sample (n=3).
2. Results
2.1 Self-Assembling of Lipopeptides Prepared via Solubilisation in Culture Medium

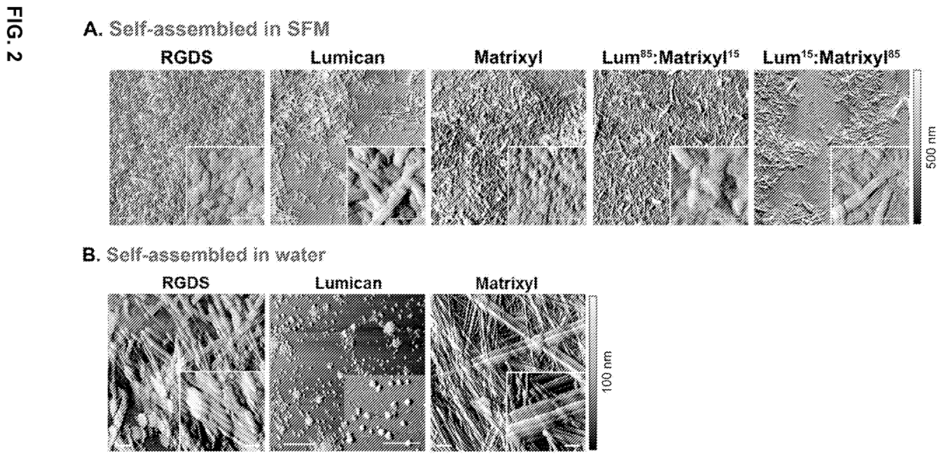

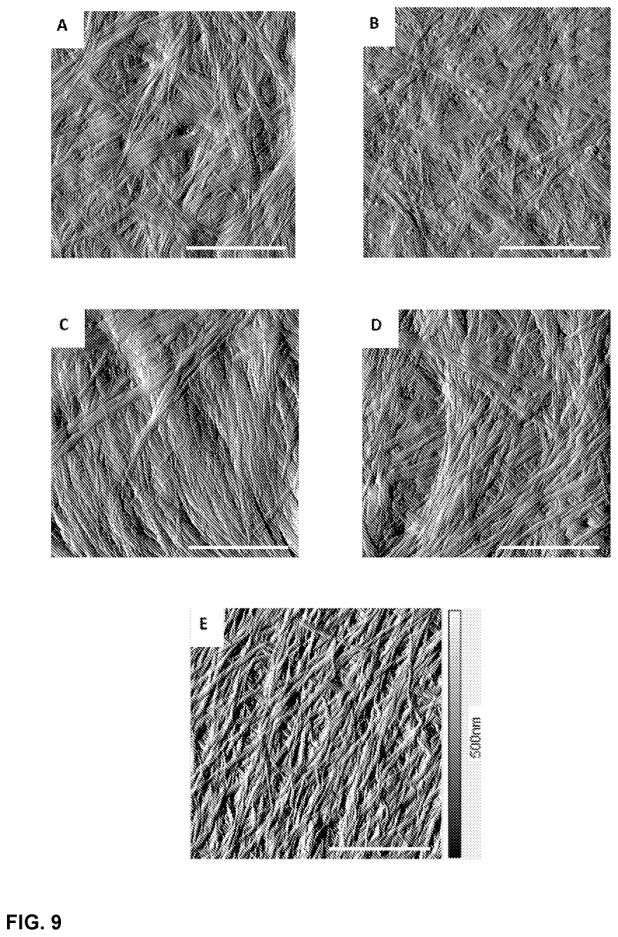

When the lipopeptides (lipidated peptides) are solubilised in serum-free culture medium (SFM) rather than double distilled water (as they normally are), their self-assembly occurs assuming a different supramolecular nanostructure (FIG. 2 and FIG. 9).

Results show that single-type RGDS lipopeptide self-assembled in medium quite differently compared to water. In water, RGDS lipopeptide forms networks of individual nanotapes/twisted nanotapes (Castelletto et al., 2013; Gouveia et al., 2013). On the other hand, in SFM such lipopeptide form bigger and denser arrangements comprising fused nanotapes. Similarly, single-type Lumican and Matrixyl lipopeptides self-assembled in SFM quite differently compared to water (FIG. 2). In water, Lumican lipopeptide assembled in borderline structures between lipid-like and amyloid peptide-like self-assembly (i.e., between nanotape and fibril structures) (Hamley et al., 2015), whereas once self-assembled Matrixyl presented wide nanotapes (Castelletto et al., 2010). In contrast, Lumican and Matrixyl lipopeptides form fused fibrillar structures in SFM, arranged in dense aggregates. Finally, the composite systems Lum⁸⁵:Matrixyl¹⁵ and Lum¹⁵:Matrixyl⁸⁵ formed structures resembling more the single-type Lumican lipopeptide than the single-type Matrixyl lipopeptide. Altogether, these results show the presence of supramolecular self-assembled structures of the single-type and composite lipopeptides in medium and a striking difference in structure when compared to structures assembled in water.
2.2 Effect of the Cell Culture Medium on Lipopeptides Biocompatibility and Bioactivity

The effect of the different nanostructure on biocompatibility and bioactivity was investigated next. For this study, the lipopeptides were used as a media supplement, rather than coating. The bioactivity of the lipopeptides has been evaluated for three different cell types, namely human stromal progenitor cells, human adipose-derived mesenchymal stem cells (hASCs) and immortalized mouse myoblast cell line (C2C12).
2.2.1 Effect on Human Stromal Progenitor Cells

The inventors hypothesised that the effect exerted by the same lipopeptide molecule can be different depending on the solvent in which the self-assembly is initiated. Lumican, Matrixyl and RGDS lipopeptides were investigated as supplements to culture media using stock solutions which were previously self-assembled either in water or in medium. Specifically, cells were cultured for 7 days and proliferation was assessed at day 3 and 7, whereas collagen deposition was analysed at day 7.

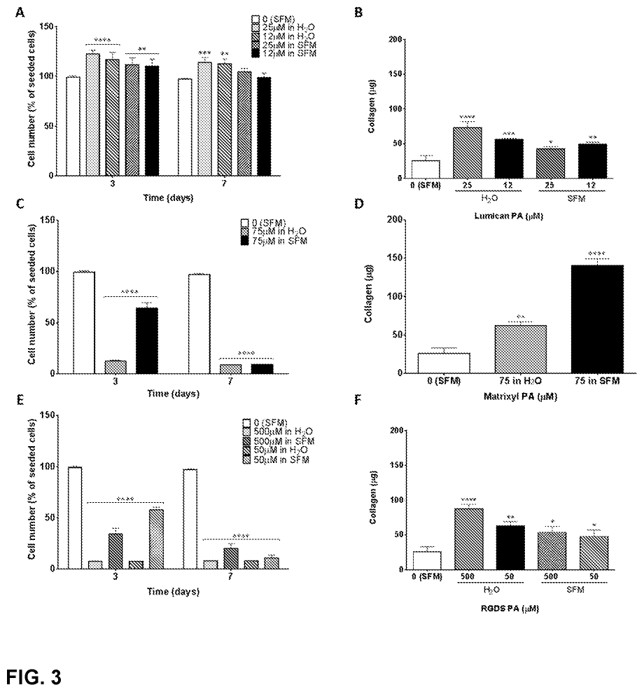

Results showed that Lumican lipopeptide significantly increased cell proliferation when self-assembled in water and supplemented at 25 μM (25 μM H₂O) and 12 μM (12 μM H₂O) compared to non-supplemented serum free medium (SFM) both at day 3 (p<0.0001) and 7 (p=0.001 and p=0.0005 respectively) (FIG. 3A).

On the other hand, Lumican lipopeptide self-assembled in medium and supplemented at the same concentrations, slightly increased cell proliferation only up to day 3 (p=0.003 and p=0.0012 for 12 and 25 μM, respectively) (FIG. 3A).

Similarly, the amount of collagen deposited by human stromal progenitor cells cultured for 7 days with Lumican lipopeptide self-assembled in water was ≈75% and 15% higher at 25 μM and 12 μM respectively, compared to their corresponding concentrations of PA self-assembled in medium (FIG. 3B).

Nonetheless, the presence of the lipopeptide significantly increased collagen deposition compared to the control (SFM) independently of the solvent in which the self-assembly occurred. In contrast, Matrixyl lipopeptide self-assembled in SFM was found to: i) dramatically decreased cell viability at day 7 in both conditions (p<0.0001) compared to the control (FIG. 3C) and ii) significantly increased the amount of deposited collagen at day 7 compared to the control (p<0.0001) and was 55% higher compared to Matrixyl lipopeptide self-assembled in water (FIG. 3D).

Furthermore, RGDS lipopeptide self-assembled in water was found to greatly impair cell proliferation compared to RGDS lipopeptide self-assembled in medium at both concentrations (50 and 500 μM), with both conditions and concentrations significantly lower to the control (p <0.0001) at day 3 and 7 (FIG. 3E). However, both conditions and concentrations significantly increased the amount of deposited collagen compared to the control, with RGDS lipopeptide self-assembled in water increasing the amount of collagen of 65% and 35% at 500 μM and 50 μM respectively, compared to their corresponding concentrations of lipopeptide self-assembled in medium (FIG. 3F). Altogether, results reported in FIG. 3 indicate that the solvent in which the self-assembly initially occurs has a lasting effect on the biocompatibility and bioactivity of the lipopeptide on human stromal progenitor cell.
2.2.3 Effect on Human Adipose-Derived Mesenchymal Stem Cells (hASCs)

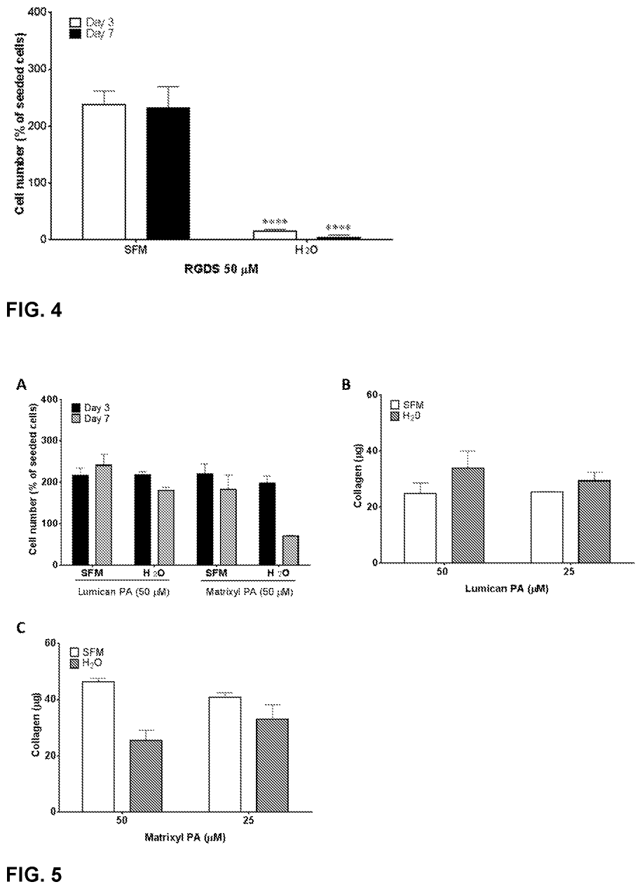

The hASCs were also cultured with RGDS lipopeptide previously solubilised above its c.a.c. in water and then diluted in SFM at the final concentration of 50 μM (FIG. 4). This was to understand if the different self-assembled structures affected hASCs behaviour as found for human stromal progenitor cell. Results showed that RGDS self-assembled in water had indeed a statistically significant (p<0.0001 since day 3) toxic effect on cell proliferation that RGDS lipopeptide self-assembled in medium did not display (comparable to FIG. 3E).

Moreover, hASCs were also cultured with Lumican or Matrixyl lipopeptides previously solubilised above their c.a.c. in water and then diluted in SFM at the final concentration of 50 μM (FIG. 5). Although not significant, results showed that both lipopeptides slightly increase cell proliferation when solubilised in medium rather than water (FIG. 5A). Significant differences in collagen deposition were not present, however, Lumican lipopeptide (FIG. 5B) in water and Matrixyl lipopeptide (FIG. 5C) in SFM slightly increased the collagen deposited by hASCs after 7 days in culture.
2.2.3 Effect on Immortalized Mouse Myoblast Cell Line (C2C12)

C2C12 were cultured up to 7 days in serum-free medium (SFM) supplemented either with Lumican or Matrixyl or RGDS lipopeptides. Each lipopeptide stock was prepared above its c.a.c. either in serum-free medium (SFM) or in water (H2O). C2C12 proliferation was evaluated using Alamar Blue assay at day 3 and 7. The amount of deposited collagen was quantified using the Sirius Red assay at day 7.

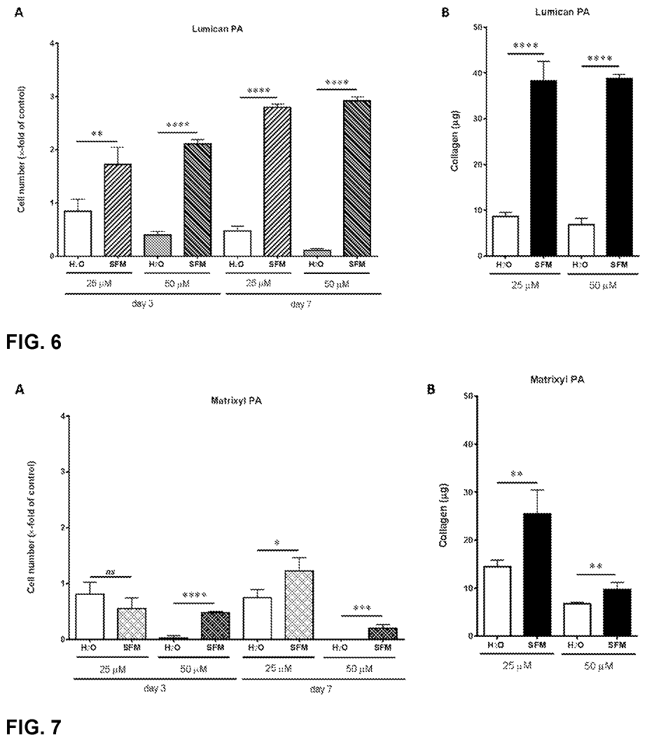

Lumican lipopeptide was tested at 25 and 50 μM and results showed that when solubilised in medium significantly increased cell proliferation since day 3 (FIG. 6). Moreover, the collagen deposited significantly increased when such lipopeptide was solubilised in SFM rather than water (FIG. 6).

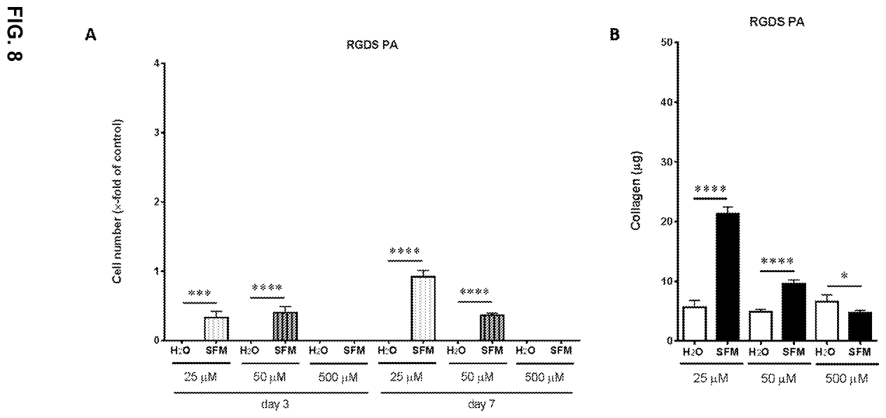

Similarly, Matrixyl lipopeptide was tested at 25 and 50 μM and both cell proliferation and collagen deposition significantly increased when the lipopeptide was solubilised in medium (FIG. 7).

Finally, RGDS lipopeptide was tested at 25, 50 and 500 μM and results showed as such lipopeptide has a quite strong toxic effect on C2C12 when solubilised in water (FIG. 8). On the other hand, when solubilised in SFM it significantly reduced its toxic effect and increased collagen deposition (FIG. 8).

### REFERENCES

Castelletto, V., Hamley, I. W., Perez, J., Abezgauz, L. and Danino, D. (2010) ‘Fibrillar superstructure from extended nanotapes formed by a collagen-stimulating peptide’, Chemical Communications, 46(48), pp. 9185-9187.

Gouveia, R. M., Castelletto, V., Alcock, S. G., Hamley, I. W. and Connon, C. J. (2013) ‘Bioactive films produced from self-assembling peptide amphiphiles as versatile substrates for tuning cell adhesion and tissue architecture in serum-free conditions’, Journal of Materials Chemistry B, 1(44), pp. 6157-6169

Jimenez, W., Pares, A., Caballeria, J., Heredia, D., Bruguera, M., Torres, M., Rojkind, M. and Rodes, J. (1985) ‘Measurement of fibrosis in needle liver biopsies: evaluation of a colorimetric method’, Hepatology, 5(5), pp. 815-8.

Hamley, I. W., Dehsorkhi, A., Castelletto, V., Walter, M. N. M., Connon, C. J., Reza, M. and Ruokolainen, J. (2015) ‘Self-Assembly and Collagen-Stimulating Activity of a Peptide Amphiphile Incorporating a Peptide Sequence from Lumican’, Langmuir, 31(15), pp. 4490-4495

1. A supramolecular structure comprising a plurality of fused fibrils wherein each fibril comprises a plurality of cell adhesion motif lipopeptides.

2. The structure of claim 1 wherein the cell adhesion motif is an extracellular matrix protein sequence or a fragment or a variant thereof.

3. The structure of claim 2, wherein the extracellular matrix protein is selected from the group consisting of fibronectin, collagen, lumican, decorin, laminin, vitronectin, fibrinogen, elastin, biglycan, heparin, tenascin and osteopontin.

4. The structure of claim 2, wherein the cell adhesion motif is chosen from the group consisting of (a)-(d), wherein (a)-(d) are:
a) a fibronectin fragment comprising an amino acid sequence selected from RGD (SEQ ID NO: 1), RGDS (SEQ ID NO: 5), PHSRN (SEQ ID NO: 6), LDVP (SEQ ID NO: 7), WQPPRARI (SEQ ID NO: 8), IGD (SEQ ID NO: 9), REDV (SEQ ID NO: 10), and IDAP (SEQ ID NO: 11) or a variant thereof;
b) a collagen fragment comprising an amino acid sequence selected from KTTKS (SEQ ID NO:2), GTPGPQGIAGQRGVV (SEQ ID NO: 12), GROGER (SEQ ID NO: 13), GLKGEN (SEQ ID NO: 14), GFOGER (SEQ ID NO: 15), and MNYYSNS (SEQ ID NO: 16) or a variant thereof;
c) a lumican fragment comprising an amino acid sequence selected from EVTLN (SEQ ID NO: 17), ELDLSYNKLK (SEQ ID NO: 18) and YEALRVANEVTLN (SEQ ID NO: 3); and
d) a laminin fragment comprising an amino acid sequence selected from the YIGSR (SEQ ID NO: 19), IKVAV (SEQ ID NO: 20), CCRRIKVAVWLC (SEQ ID NO: 21) and RGD.

5. The structure of claim 1, wherein the plurality of cell adhesion motif lipopeptides comprise at least two different cell adhesion motif lipopeptides.

6. The structure of claim 5, wherein the at least two different cell adhesion motif lipopeptides are a cell adhesion motif lipopeptide comprising KTTKS (SEQ ID NO:2) and a cell adhesion motif lipopeptide comprising YEALRVANEVTLN (SEQ ID NO: 3).

7. The structure of claim 1, wherein the lipopeptide comprises a lipid portion comprising a carbon chain of 6 to 24 carbon atoms.

8. An aqueous medium comprising a supramolecular structure in accordance with claim 1.

9. The medium of claim 8, wherein the medium is a cell culture medium.

10. The medium of claim 9, wherein the cell culture medium is serum free.

11. The medium of claim 9, wherein the cell culture medium is selected from the group consisting of Dulbecco's Modified Eagle Medium (DMEM), Ham's F12, Leibovitz's L-15 medium, RPMI-1640, Mesencult™ Basal Medium, or DMEM-F12.

12. A surface for cell maintenance, cell culture or cell bioprocessing, wherein immobilised in or on the surface is the supramolecular structure of claim 1.

13. The surface of claim 12, wherein the surface is 2D or 3D.

14. The surface of claim 13, wherein the surface is 2D and the 2D surface is a cover slip or a surface of a cell culture vessel, optionally wherein the cell culture vessel is selected from a tube, a flask, a dish or a plate comprising a plurality of wells.

15. The surface of claim 13, wherein the surface is 3D and the 3D surface is a scaffold, optionally wherein the scaffold is a hydrogel or a polystyrene scaffold.

16. A method for maintenance, culture, or bioprocessing of a cell using one or more of the supramolecular structure of claim 1 the aqueous medium of claim 8, or the surface of claim 12.

17. The method according to claim 16, wherein the bioprocessing is for collagen production.

18. The method according to claim 16, wherein the supramolecular structure, medium or surface promotes cell growth.

19. The method of claim 16, wherein the cell is selected from the group consisting of: a human stromal progenitor cell, a human adipose derived mesenchymal stem cell and an immortalised mouse myoblast cell.

20. A method of producing a supramolecular structure comprising a plurality of fused fibrils wherein each fibril comprises a plurality of cell adhesion motif lipopeptides, the method comprising self-assembly of the plurality of cell adhesion motif lipopeptides in an aqueous medium having an ionic strength that is greater than the ionic strength of distilled water to produce the supramolecular structure.

21. The method of claim 20, wherein the method comprises dissolving lyophilised lipopeptides in the aqueous medium having an ionic strength that is greater than the ionic strength of distilled water, optionally wherein the aqueous medium is serum-free cell culture medium.

22. The method of claim 21, Dulbecco's Modified Eagle Medium (DMEM), Ham's F12, Leibovitz's L-15 medium, RPMI-1640, Mesencult™ Basal Medium, and DMEM-F12.
